(19) United States
(12) Patent Application Publication
(10) Pub. No.: US 2014/0180147 A1
(43) Pub. Date: Jun. 26, 2014

(54) ESTIMATING INTERSPLINE DISTANCES ON MAPPING CATHETERS

(71) Applicant: Boston Scientific Scimed, Inc., Maple Grove, MN (US)

(72) Inventors: Pramodsingh H. Thakur, Woodbury, MN (US); Barun Maskara, Blaine, MN (US); Allan C. Shuros, St. Paul, MN (US); Shantha Arcot-Krishnamurthy, Renton, WA (US); Shibaji Shome, Arden Hills, MN (US); Sunipa Saha, Shoreview, MN (US)

(73) Assignee: Boston Scientific Scimed, Inc., Maple Grove, MN (US)

(21) Appl. No.: 14/136,583

(22) Filed: Dec. 20, 2013

Related U.S. Application Data
(60) Provisional application No. 61/739,970, filed on Dec. 20, 2012.

(51) Int. Cl. A61B 19/00; A61B 5/02

(52) U.S. Cl. CPC A61B 19/46; A61B 5/02
USPC 600/508

(57) ABSTRACT

A catheter system includes a mapping catheter having a plurality of splines, each of the plurality of splines including a plurality of mapping electrodes. The system further includes a processor operatively coupled to the plurality of mapping electrodes and configured to receive signals sensed by the plurality of mapping electrodes. The processor is further configured to estimate an interspline distance between adjacent splines in the plurality of splines based on the signals sensed by the mapping electrodes on the adjacent splines.

### CROSS-REFERENCE TO RELATED APPLICATION

This application claims the benefit of Provisional Application No. 61/739,970, filed Dec. 20, 2012, which is incorporated herein by reference in its entirety.

### TECHNICAL FIELD

The present disclosure relates to mapping systems. More particularly, the present disclosure relates to a mapping system configured to estimate interspline distances on basket mapping catheters.

### BACKGROUND

Physicians make use of catheters in medical procedures to gain access into interior regions of the body for diagnostic and therapeutic purposes. It is important for the physician to be able to precisely position the catheter within the body to gain contact with a desired tissue location. During these procedures, a physician steers the catheter through a main vein or artery into the interior region of the heart that is to be treated. The physician then further manipulates a steering mechanism to place the electrode carried on the distal tip of the catheter into direct contact with the endocardial tissue. The physician directs energy from the electrode through myocardial tissue either to an indifferent electrode (in a unipolar electrode arrangement) or to an adjacent electrode (in a bi-polar electrode arrangement) to ablate the tissue.

Before ablating heart tissue, physicians often examine the propagation of electrical impulses in heart tissue to locate aberrant conductive pathways and to identify the arrhythmia foci, which are ablated. The techniques used to analyze these pathways and locate foci are commonly called mapping.

### SUMMARY

Disclosed herein are various embodiments of methods for determining interspline distances on a mapping catheter including a plurality of splines, as well as cardiac mapping systems employing such methods.

In Example 1, a catheter system includes a mapping catheter having a plurality of splines, each of the plurality of splines including a plurality of mapping electrodes. The system further includes a processor operatively coupled to the plurality of mapping electrodes and configured to receive signals sensed by the plurality of mapping electrodes. The processor is further configured to estimate an interspline distance between adjacent splines in the plurality of splines based on the signals sensed by the mapping electrodes.

In Example 2, the catheter system according to Example 1, wherein the processor is configured to estimate the interspline distance based on a comparison of signals sensed by intraspline mapping electrode with signals sensed by mapping electrodes on adjacent splines.

In Example 3, the catheter system according to either Example 1 or Example 2, wherein the processor is configured to estimate the interspline distance based on a comparison of latency between signals sensed by the intraspline mapping electrodes with latency between signals sensed by interspline mapping electrodes.

In Example 4, the catheter system according to any of Examples 1-3, wherein the processor is configured to estimate the interspline distance based on a comparison of impedance between the intraspline mapping electrodes with impedance between interspline mapping electrodes.

In Example 5, the catheter system according to any of Examples 1-4, wherein the processor is configured to estimate the interspline distance based on a comparison of signals sensed by a first interspline pair of mapping electrodes with a second interspline pair of mapping electrodes.

In Example 6, the catheter system according to any of Examples 1-5, wherein the processor is further configured to interpolate the interspline distance along portions of the adjacent splines between the first interspline pair of mapping electrodes and the second interspline pair of mapping electrodes.

In Example 7, the catheter system according to any of Examples 1-6, and further comprising a display associated with the processor and configured to display a representation of the mapping catheter, wherein the displayed representation of the mapping catheter is based on interspline distances estimated by the processor.

In Example 8, a method for estimating interspline distances on a mapping catheter having a plurality of splines, each of the plurality of splines including a plurality of mapping electrodes, includes measuring a parameter between a first pair of mapping electrodes among the plurality of mapping electrodes, measuring the parameter between a second pair of mapping electrodes among the plurality of mapping electrodes, and determining the interspline distances based on a comparison of the parameter measured by the first pair of mapping electrodes with the parameter measured by the second pair of mapping electrodes.

In Example 9, the method according to Example 8, wherein the first pair of mapping electrodes are intraspline electrodes and the second pair of mapping electrodes are interspline electrodes.

In Example 10, the method according to either Example 8 or Example 9, wherein the parameter is latency, and wherein the determining step comprises estimating the interspline distance based on a comparison of latency between signals sensed by the first, intraspline pair of mapping electrodes with latency between signals sensed by second, interspline pair of mapping electrodes.

In Example 11, the method according to any of Examples 8-10, wherein the parameter is impedance, and wherein the determining step comprises estimating the interspline distance based on a comparison of impedance measured between the first, intraspline pair of mapping electrodes with impedance between the second, interspline pair of mapping electrodes.

In Example 12, the method according to any of Examples 8-11, wherein the first and second pairs of mapping electrodes are interspline electrodes, and wherein the determining step comprises estimating the interspline distance based on a comparison of signals sensed by the first pair of mapping electrodes with the second pair of mapping electrodes

In Example 13, the method according to any of Examples 8-12, wherein the determining step further comprises interpolating the interspline distance along portions of the adjacent splines between the first pair of mapping electrodes and the second pair of mapping electrodes.

In Example 14, the method according to any of Examples 8-13, displaying a representation of the mapping catheter, wherein the displayed representation of the mapping catheter is based on the determined interspline distances.

In Example 15, a method for displaying a mapping catheter in a mapping system, the mapping catheter including a plurality of splines, each of the plurality of splines including a plurality of mapping electrodes, the method including positioning the plurality of mapping electrodes proximate to an anatomical structure, sensing signals with the plurality of mapping electrodes, estimating an interspline distance between adjacent splines in the plurality of splines based on the signals sensed by the mapping electrodes on the adjacent splines, and displaying a representation of the mapping catheter according to the estimated interspline distances.

In Example 16, the method according to Example 15, wherein the estimating step comprises measuring a parameter between a first pair of mapping electrodes among the plurality of mapping electrodes, measuring the parameter between a second pair of mapping electrodes among the plurality of mapping electrodes, and determining the interspline distances based on a comparison of the parameter measured by the first pair of mapping electrodes with the parameter measured by the second pair of mapping electrodes.

In Example 17, the method according to either Example 15 or Example 16, wherein the estimating step comprises estimating the interspline distance based on a comparison of latency between signals sensed by an intraspline pair of mapping electrodes with latency between signals sensed by an interspline pair of mapping electrodes.

In Example 18, the method according to any of Examples 15-17, wherein the estimating step comprises estimating the interspline distance based on a comparison of impedance measured between an intraspline pair of mapping electrodes with impedance between an interspline pair of mapping electrodes.

In Example 19, the method according to any of Examples 15-18, wherein estimating step comprises estimating the interspline distance based on a comparison of signals sensed by a first interspline pair of mapping electrodes with the second interspline pair of mapping electrodes.

In Example 20, the method according to any of Examples 15-19, wherein the estimating step further comprises interpolating the interspline distance along portions of the adjacent splines between the first interspline pair of mapping electrodes and the second interspline pair of mapping electrodes.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic view of an embodiment of a system for accessing a targeted tissue region in the body for diagnostic and therapeutic purposes.

FIG. 2 is a schematic view of an embodiment of a mapping catheter having a basket functional element carrying structure for use in association with the system of FIG. 1.

FIG. 3 is a schematic side view of an embodiment of the basket functional element including a plurality of mapping electrodes.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

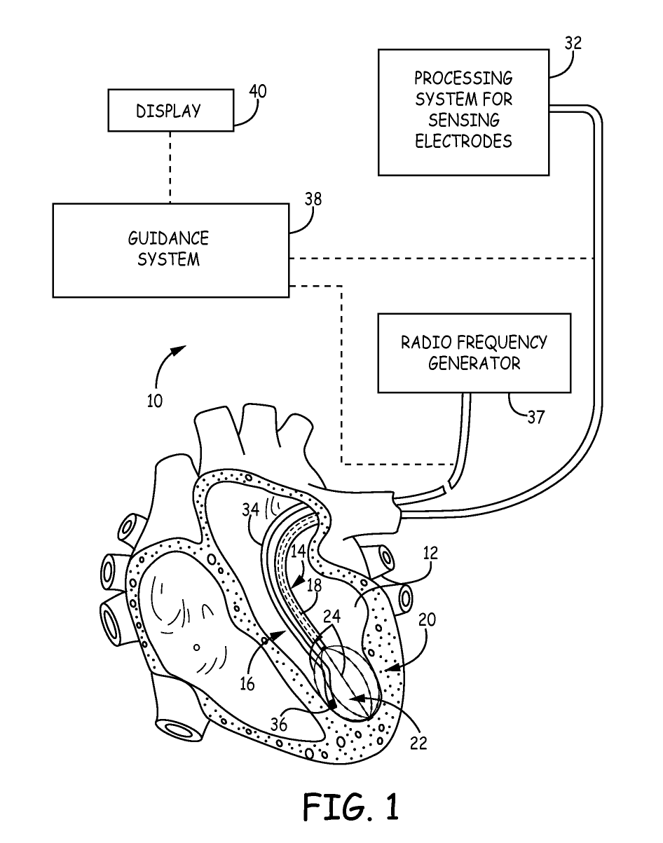

FIG. 1 is a schematic view of a system 10 for accessing a targeted tissue region in the body for diagnostic or therapeutic purposes. FIG. 1 generally shows the system 10 deployed in the left ventricle of the heart. Alternatively, system 10 can be deployed in other regions of the heart, such as the left atrium, right atrium, or right ventricle. While the illustrated embodiment shows the system 10 being used for ablating heart tissue, the system 10 (and the methods described herein) may alternatively be configured for use in other tissue ablation applications, such as procedures for ablating tissue in the prostrate, brain, gall bladder, uterus, and other regions of the body, including in systems that are not necessarily catheter-based.

The system 10 includes a mapping probe 14 and an ablation probe 16. In FIG. 1, each is separately introduced into the selected heart region 12 through a vein or artery (e.g., the femoral vein or artery) through suitable percutaneous access. Alternatively, the mapping probe 14 and ablation probe 16 can be assembled in an integrated structure for simultaneous introduction and deployment in the heart region 12.

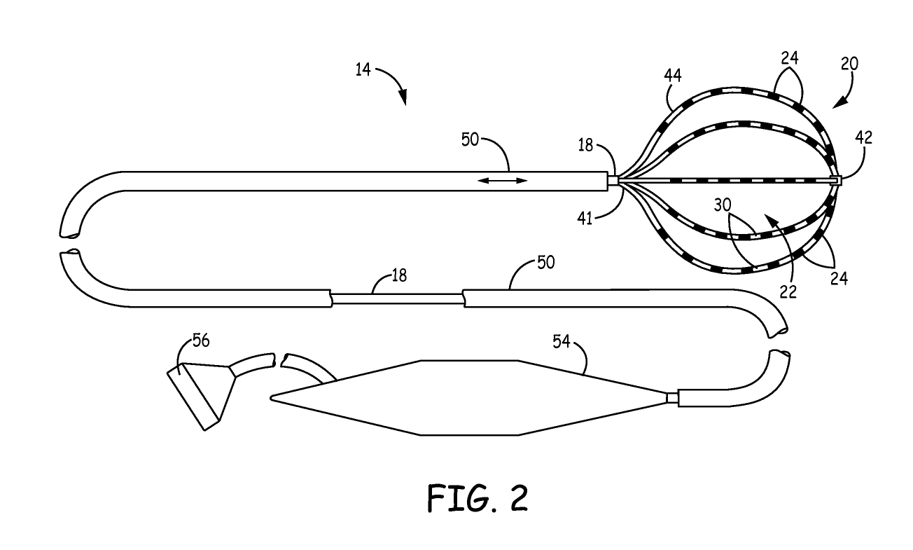

The mapping probe 14 has a flexible catheter body 18. The distal end of the catheter body 18 carries a three-dimensional multiple electrode structure 20. In the illustrated embodiment, the structure 20 takes the form of a basket defining an open interior space 22 (see FIG. 2), although other multiple electrode structures could be used. The multiple electrode structure 20 carries a plurality of electrodes 24 configured to sense intrinsic physiological activity in the anatomical region on which the ablation procedure is to be performed.

The electrodes 24 are electrically coupled to a processing system 32. A signal wire (not shown) is electrically coupled to each electrode 24 on the basket structure 20. The wires extend through the body 18 of the probe 14 and electrically couple the electrodes 24 to the processing system 32 and the guidance system 34. The electrodes 24 sense intrinsic electrical activity in heart tissue. The sensed activity is processed by the processing system 32 to assist the physician in identifying the site or sites within the heart appropriate for ablation.

In some embodiments, the processing system 32 may be configured to measure the intrinsic electrical activity in the heart tissue adjacent to the electrodes 24. For example, in some embodiments, the processing system 32 is configured to detect intrinsic electrical activity associated with a dominant rotor in the anatomical feature being mapped. Studies have shown that dominant rotors have a role in the initiation and maintenance of atrial fibrillation, and ablation of the rotor path and/or rotor core may be effective in terminating the atrial fibrillation. The processing system 32 processes the sensed information to derive the location of a site appropriate for ablation using the ablation probe 16.

The ablation probe 16 includes a flexible catheter body 34 that carries one or more ablation electrodes 36. The one or more ablation electrodes 36 are electrically connected to a radio frequency generator 37 that is configured to deliver ablation energy to the one or more ablation electrodes 36. The ablation probe 16 is movable with respect to the anatomical feature to be treated, as well as the structure 20. The ablation probe 16 is positionable between or adjacent to electrodes 24 of the structure 20 as the one or more ablation electrodes 36 are positioned with respect to the tissue to be treated.

A guidance system 38 is electrically coupled to the mapping catheter 14 and the ablation catheter 16. The guidance system 38 collects and processes information regarding the location of the ablation probe 16 within the space 22 defined by the basket structure 20, in term of its position relative to the position of the electrodes 24. The guidance system 38 provides a position-identifying output that aids the physician in guiding the ablation electrodes 36 into contact with tissue at the site identified for ablation. The guidance system 38 can process and provide position-specific information in various ways.

In the illustrated embodiment, the guidance system 38 includes an output display device 40 (e.g., a CRT, LED display, or a printer). In some embodiments, the display device 40 is configured to display a representation of the mapping catheter 14 and ablation catheter 16 relative to the anatomical structure. As will be described in more detail herein, the display device 40 is configured to update the display of the mapping catheter 14 based on an estimation of the interspline distance of the mapping catheter 14.

FIG. 2 illustrates an embodiment of the mapping catheter 14 including electrodes 24 at the distal end suitable for use in the system 10 shown in FIG. 1. The mapping catheter 14 has a flexible catheter body 18, the distal end of which carries the three dimensional structure 20 configured to carry the mapping electrodes or sensors 24. The mapping electrodes 24 sense intrinsic electrical activity in the heart tissue, which sensed activity is then processed by the processing system 32 and guidance system 38 to assist the physician in identifying the site or sites having a heart rhythm disorder. This process is commonly referred to as mapping. This information can then be used to determine an appropriate location for applying appropriate therapy (e.g., ablation) to the identified sites, and to navigate the one or more ablation electrodes 36 to the identified sites.

The illustrated three dimensional structure 20 comprises a base member 41 and an end cap 42 between which flexible splines 44 generally extend in a circumferentially spaced relationship. As discussed above, the three dimensional structure 20 takes the form of a basket defining an open interior space 22. In some embodiments, the splines 44 are made of a resilient inert material, such as, e.g., Nitinol metal or silicone rubber, and are connected between the base member 41 and the end cap 42 in a resilient, pretensed condition, to bend and conform to the tissue surface they contact. In the illustrated embodiment, eight splines 44 form the three dimensional structure 20. Additional or fewer splines 44 could be used in other embodiments. As illustrated, each spline 44 carries eight mapping electrodes 24. Additional or fewer mapping electrodes 24 could be disposed on each spline 44 in other embodiments of the three dimensional structure 20. In the illustrated embodiment, the three dimensional structure 20 is relatively small (e.g., 40 mm or less in diameter). In alternative embodiments, the three dimensional structure 20 is larger (e.g., 40 mm in diameter or greater).

The three dimensional structure 20 generally forms a basket shape with regularly spaced splines 44 when the three dimensional structure 20 is in expanded outside of the anatomical structure. However, when the three dimensional structure 20 is expanded within the confines of the anatomical structure to be mapped and treated, the splines 44 may not be distributed uniformly due to the non-uniform shape of the anatomical structure and the flexibility of the splines 44. As will be described in more detail herein, the system 10 is configured to calculate interspline distances when the three dimensional structure 20 is disposed in the anatomical structure to assure accurate characterization of the intrinsic physiological activity sensed by the mapping electrodes 24.

A slidable sheath 50 is movable along the major axis of the catheter body 30. Moving the sheath 50 forward (i.e., toward the distal end) causes the sheath 50 to move over the three dimensional structure 20, thereby collapsing the structure 20 into a compact, low profile condition suitable for introduction into an interior space, such as, for example, into the heart. In contrast, moving the sheath 50 rearward (i.e., toward the proximal end) frees the three dimensional structure 20, allowing the structure 20 to spring open and assume the pretensed position illustrated in FIG. 2. Further details of embodiments of the three dimensional structure 20 are disclosed in U.S. Pat. No. 5,647,870, entitled “Multiple Electrode Support Structures,” which is hereby incorporated by reference in its entirety.

A signal wire (not shown) is electrically coupled to each mapping electrode 26. The wires extend through the body 30 of the mapping catheter 20 into a handle 54, in which they are coupled to an external connector 56, which may be a multiple pin connector. The connector 56 electrically couples the mapping electrodes 24 to the processing system 32 and guidance system 38. Further details on mapping systems and methods for processing signal generated by the mapping catheter are discussed in U.S. Pat. No. 6,070,094, entitled “Systems and Methods for Guiding Movable Electrode Elements within Multiple-Electrode Structure,” U.S. Pat. No. 6,233,491, entitled “Cardiac Mapping and Ablation Systems,” and U.S. Pat. No. 6,735,465, entitled “Systems and Processes for Refining a Registered Map of a Body Cavity,” the disclosures of which are incorporated herein by reference.

It is noted that other multi-electrode structures could be deployed on the distal end. It is further noted that the multiple mapping electrodes 24 may be disposed on more than one structure rather than, for example, the single mapping catheter 14 illustrated in FIG. 2. For example, if mapping within the left atrium with multiple mapping structures, an arrangement comprising a coronary sinus catheter carrying multiple mapping electrodes and a basket catheter carrying multiple mapping electrodes positioned in the left atrium may be used. As another example, if mapping within the right atrium with multiple mapping structures, an arrangement comprising a decapolar catheter carrying multiple mapping electrodes for positioning in the coronary sinus, and a loop catheter carrying multiple mapping electrodes for positioning around the tricuspid annulus may be used.

Additionally, although the mapping electrodes 24 have been described as being carried by dedicated probes, such as mapping catheter 14, the mapping electrodes can be carried on non-mapping dedicated probes. For example, an ablation catheter (e.g., the ablation catheter 16) can be configured to include one or mapping electrodes 24 disposed on the distal end of the catheter body and coupled to the signal processing system 32 and guidance system 38. As another example, the ablation electrode at the distal end of the ablation catheter may be coupled to the signal processing system 32 and guidance system 38 to also operate as a mapping electrode.

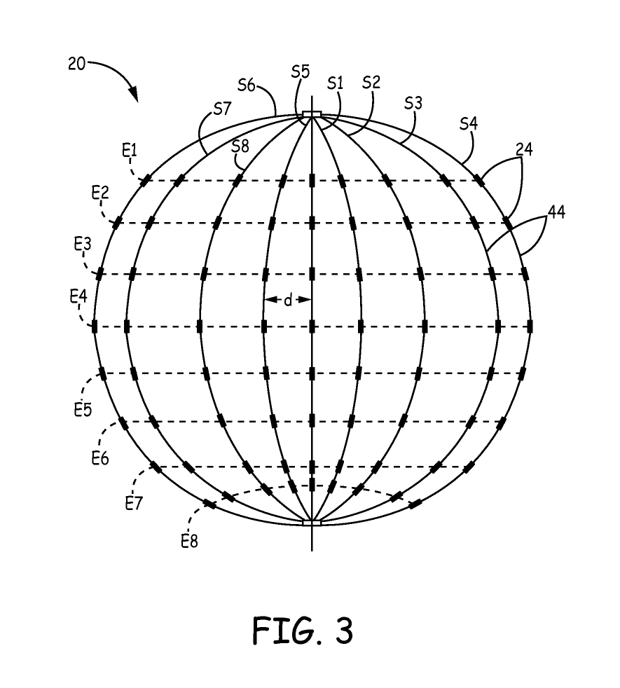

To illustrate the operation of the system 10, FIG. 3 is a schematic view of an embodiment of the basket structure 20 including a plurality of mapping electrodes 24 disposed on a plurality of splines 44. In the illustrated embodiment, the basket structure includes 64 mapping electrodes 24. The mapping electrodes 24 are disposed in groups of eight electrodes 24 (labeled E1, E2, E3, E4, E5, E6, E7, and E8) on each of eight splines 44 (labeled S1, S2, S3, S4, S5, S6, S7, and S8). While the sixty-four mapping electrodes 24 are shown disposed on a basket structure 20, the mapping electrodes 24 may alternatively be arranged in different numbers and on different structures.

As discussed above, when the basket structure 20 is positioned adjacent to an anatomical structure, the splines 44 may move with respect to each other to conform to the contours of the anatomical structure. As a result, the distance d between adjacent splines 44 may be non-uniform across the basket structure 20. According to embodiments of the present disclosure, the interspline distance d between each of the splines 44 is determined to ensure that the system 10 can generate an accurate representation of the intrinsic physiological signals of the anatomical structure, and can display a correct representation of the basket structure 20 (e.g., on the display 40).

When the basket structure 20 is disposed adjacent to the anatomical structure, the processing system 32 receives signals sensed by the electrodes 24. In order to determine the distance d between adjacent splines, the processing system 32 may measure one or more parameters based on the signals sensed by the electrodes 24, and then extrapolate this information to estimate the distance d. In some embodiments, the processing system 32 is configured to measure the one or more parameters between electrodes 24 on the same spline 44 and between electrodes 24 on different splines 44. The electrodes 24 on the same spline are a known distance apart, to the processing system 32 can use this information to extrapolate the distance to another spline 44 (e.g., an adjacent spline 44) based on the measured one or more parameters. In some embodiments, the one or more parameters measured by electrodes 24 may be averaged with other nearby electrodes 24 to improve the accuracy of the parameter measurements.

In some embodiments, the processing system 32 can measure the latency of the signals between electrodes 24 on the same spline and compare this latency with the latency of the signals between electrodes 24 on adjacent splines. For example, if the latency between the electrode E5 on the spline S2 and the electrode E5 on the spline S3 is measured by the processing system 32 to be 5 milliseconds (ms) and the latency between the electrodes E4 and E5 on the spline S2, which are known to be 2 millimeters (mm) apart, is measured by the processing system 32 to be 3 ms, then the processing system can estimate the distance between the splines S2 and S3 to be approximately 3.33 mm. In some embodiments, the conduction velocity between electrodes can be bound by physiological limits to assure accuracy of the latency measurements. In some embodiments, the latency is compared across multiple pairs of electrodes simultaneously to correct to anomalies in the latency measurements.

Alternatively, the processing system 32 can measure the latency of signals between one interspline electrode pair and compare this measurement with a measured latency between another interspline electrode pair. For example, in some embodiments, the latency may be measured between a first cross-spline pair of electrodes 24 (e.g., the electrodes E5 on the splines S1 and S2) and between a second cross-spline pair of electrodes 24 (e.g., the electrodes E5 on splines S2 and S3) to determine a relative distance between the splines 44 at the measured cross-spline electrodes 24. This measurement can be performed for some or all splines 44 to determine the relative distances between adjacent splines 44 at the location of the electrodes 24 used in the cross-spline measurements. This information can then be interpolated to estimate the relative positioning of the splines 44.

As another example, in some embodiments, the processing system 32 can measure the impedance between electrodes 24 on the same spline and compare this impedance with the impedance between electrodes 24 on adjacent splines 44. For example, the impedance between the electrode E5 on the spline S2 and the electrode E5 on the spline S3 can be measured and compared to the impedance between the electrodes E4 and E5 on the spline S2, which are a known distance apart. In some embodiments, in the event of atrial mapping, the impedance measurements are taken according to the timing of ventricular events to remove variability in measurements due to cardiac activity. Assuming impedance scales linearly with distance, the measured intraspline impedance between electrodes of known distance apart and the measured interspline impedance measurement can be used to estimate the interspline distance.

Other approaches to estimating the interspline distances of the basket structure 20 may also be employed by the processing system 32. For example, a stimulation signal (e.g., pacing signal) may be provided from one of the electrodes 24 on the basket structure 20, and the response measured at the other electrodes 24 on the basket structure 20. The decrease in artifact amplitude with increasing distance from the electrode 24 providing the stimulation signal can be used to estimate the distance between the splines 44.

Various approaches may be employed by the processing system 32 to increase the speed and efficiency of the interspline distance determination. In some embodiments, the processing system 32 is configured to measure one or more parameters with electrodes 24 at a plurality of locationally diverse parts of the basket structure 20. After estimating the interspline distance based on these measurements, the processing system 32 may then interpolate the relative positions of the splines 44 between the parameter-measuring electrodes 24. For example, in the embodiment illustrated in FIG. 3, the processing system 32 can be configured to measure one or parameters (e.g., latency or impedance) with electrodes 24 near the middle circumference of the basket structure 20 (e.g., at electrodes E4 and/or E5 on splines S1-S8) to estimate the interspline distance d between each spline S1-S8. The electrodes 24 near the middle of the basket structure 20 provide the largest variation in the non-uniformity in the interspline spacing. The processing system 32 can then measure the one or more parameters at the most proximal and/or distal electrodes E1, E8 on the splines S1-S8 to estimate the interspline distance d at the ends of the basket structure 20. With the estimated interspline distances at the middle and ends of the basket structure, the processing system 32 can interpolate the interspline distances of the splines 44 between the middle and end electrodes (i.e., at electrodes E2, E3, E6, E7 of the splines S1-S8).

When the interspline distance between each of the splines 44 has been estimated, information regarding the interspline distances can be used to update the representation of the basket structure 20 on the display device 40. The display of the basket structure 20 based on the estimated interspline distances provides the clinician with an accurate representation of the configuration of the basket structure 20 with respect to the anatomical structure being mapped by the mapping catheter 14. As a result, maps of the intrinsic physiological activity of the anatomical structure are more accurate, and the guidance system 38 can assist the clinician with precise location of the ablation electrode with respect to the basket structure 20 and identified pathologies.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A catheter system comprising:
a mapping catheter including a plurality of splines, each of the plurality of splines including a plurality of mapping electrodes;
a processor operatively coupled to the plurality of mapping electrodes and configured to receive signals sensed by the plurality of mapping electrodes, the processor further configured to estimate an interspline distance between adjacent splines in the plurality of splines based on the signals sensed by the mapping electrodes on the adjacent splines.

2. The catheter system of claim 1, wherein the processor is configured to estimate the interspline distance based on a comparison of signals sensed by intraspline mapping electrode with signals sensed by mapping electrodes on adjacent splines.

3. The catheter system of claim 2, wherein the processor is configured to estimate the interspline distance based on a comparison of latency between signals sensed by the intraspline mapping electrodes with latency between signals sensed by interspline mapping electrodes.

4. The catheter system of claim 2, wherein the processor is configured to estimate the interspline distance based on a comparison of impedance between the intraspline mapping electrodes with impedance between interspline mapping electrodes.

5. The catheter system of claim 1, wherein the processor is configured to estimate the interspline distance based on a comparison of signals sensed by a first interspline pair of mapping electrodes with a second interspline pair of mapping electrodes.

6. The catheter system of claim 5, wherein the processor is further configured to interpolate the interspline distance along portions of the adjacent splines between the first interspline pair of mapping electrodes and the second interspline pair of mapping electrodes.

7. The catheter system of claim 1, and further comprising:
a display associated with the processor and configured to display a representation of the mapping catheter, wherein the displayed representation of the mapping catheter is based on interspline distances estimated by the processor.

8. A method for estimating interspline distances on a mapping catheter including a plurality of splines, each of the plurality of splines including a plurality of mapping electrodes, the method comprising:
measuring a parameter between a first pair of mapping electrodes among the plurality of mapping electrodes;
measuring the parameter between a second pair of mapping electrodes among the plurality of mapping electrodes; and
determining the interspline distances based on a comparison of the parameter measured by the first pair of mapping electrodes with the parameter measured by the second pair of mapping electrodes.

9. The method of claim 8, wherein the first pair of mapping electrodes are intraspline electrodes and the second pair of mapping electrodes are interspline electrodes.

10. The method of claim 9, wherein the parameter is latency, and wherein the determining step comprises:
estimating the interspline distance based on a comparison of latency between signals sensed by the first, intraspline pair of mapping electrodes with latency between signals sensed by second, interspline pair of mapping electrodes.

11. The method of claim 9, wherein the parameter is impedance, and wherein the determining step comprises:
estimating the interspline distance based on a comparison of impedance measured between the first, intraspline pair of mapping electrodes with impedance between the second, interspline pair of mapping electrodes.

12. The method of claim 8, wherein the first and second pairs of mapping electrodes are interspline electrodes, and wherein the determining step comprises:
estimating the interspline distance based on a comparison of signals sensed by the first pair of mapping electrodes with the second pair of mapping electrodes

13. The method of claim 12, wherein the determining step further comprises:
interpolating the interspline distance along portions of the adjacent splines between the first pair of mapping electrodes and the second pair of mapping electrodes.

14. The method of claim 8, and further comprising:
displaying a representation of the mapping catheter, wherein the displayed representation of the mapping catheter is based on the determined interspline distances.

15. A method for displaying a mapping catheter in a mapping system, the mapping catheter including a plurality of splines, each of the plurality of splines including a plurality of mapping electrodes, the method comprising:
positioning the plurality of mapping electrodes proximate to an anatomical structure;
sensing signals with the plurality of mapping electrodes;
estimating an interspline distance between adjacent splines in the plurality of splines based on the signals sensed by the mapping electrodes on the adjacent splines; and
displaying a representation of the mapping catheter according to the estimated interspline distances.

16. The method of claim 15, wherein the estimating step comprises:
measuring a parameter between a first pair of mapping electrodes among the plurality of mapping electrodes;
measuring the parameter between a second pair of mapping electrodes among the plurality of mapping electrodes; and
determining the interspline distances based on a comparison of the parameter measured by the first pair of mapping electrodes with the parameter measured by the second pair of mapping electrodes.

17. The method of claim 15, wherein the estimating step comprises:
estimating the interspline distance based on a comparison of latency between signals sensed by an intraspline pair of mapping electrodes with latency between signals sensed by an interspline pair of mapping electrodes.

18. The method of claim 15, wherein the estimating step comprises:
estimating the interspline distance based on a comparison of impedance measured between an intraspline pair of mapping electrodes with impedance between an interspline pair of mapping electrodes.

19. The method of claim 15, wherein estimating step comprises:
estimating the interspline distance based on a comparison of signals sensed by a first interspline pair of mapping electrodes with the second interspline pair of mapping electrodes.

20. The method of claim 19, wherein the estimating step further comprises:
interpolating the interspline distance along portions of the adjacent splines between the first interspline pair of mapping electrodes and the second interspline pair of mapping electrodes.
